(19) United States
(12) Patent Application Publication
(10) Pub. No.: US 2024/0216463 A1
(43) Pub. Date: Jul. 4, 2024

(54) COMPOSITION COMPRISING COFFEE, COFFEE EXTRACT, AND BYPRODUCT AS ACTIVE INGREDIENT FOR PREVENTION OF CANCER METASTASIS OR FOR ALLEVIATION OR TREATMENT OF CANCER

(71) Applicant: DAEJEON UNIVERSITY INDUSTRY-UNIVERSITY COOPERATION FOUNDATION, Daejeon (KR)

(72) Inventors: Chang Gue SON, Daejeon (KR); Sung Bae LEE, Daejeon (KR)

(21) Appl. No.: 18/262,653

(22) PCT Filed: Jan. 20, 2022
(86) PCT No.: PCT/KR2022/001064

§ 371 Date: Jul. 24, 2023

(30) Foreign Application Priority Data
Jan. 25, 2021 (KR) 10-2021-0010455

(51) Int. Cl. A61K 36/74; A61P 35/04

(52) U.S. Cl. CPC A61K 36/74; A61P 35/04

(57) ABSTRACT

The present invention relates to a composition for preventing, alleviating or treating cancer metastasis containing coffee, a coffee extract and a coffee by-product as an active ingredient. The composition is able to inhibit cancer metastasis, cancer growth, and VCAM1 protein expression, and thus may be used to prevent or treat cancer.

### TECHNICAL FIELD

The present invention relates to a composition for preventing, alleviating or treating cancer metastasis containing coffee, a coffee extract and a coffee by-product as an active ingredient.

### BACKGROUND ART

Metastasis is the most representative and clinically important property among many properties of cancer. Metastasis occurs in most cancer patients, and over 90% of deaths in cancer patients are due to metastasis. Clinically, cancer metastasis is found in terminal cancer patients, but it has been found that many cancer cells circulate even in the blood of many cancer patients without metastasis, which means that cancer metastasis at the cellular level begins at a very early stage. In addition, as it has been found that the characteristics and tissue environment of cancer cells have a decisive effect on metastasis, the seed and soil theory that specific cancer cells are spread to and grow in specific organs has become a theory of metastasis. Thus, prevention of metastasis is the most important target in cancer treatment. However, so far, medicine has focused on the early diagnosis, early removal, and effective removal or reduction of cancer, and there have been very few studies on natural s for controlling or suppressing cancer metastasis. In addition, controlling the tumor-host microenvironment, rather than targeting the tumor itself, is emerging as a new strategy for preventing cancer metastasis.

Coffee is one of the world's most popular beverages, with over 2.25 billion cups consumed each day worldwide. In addition, coffee is a tool to maintain human relationships and wake up, and various bean types and roasting methods have become a culture that goes beyond a simple product in human life. The pharmacological properties of coffee against cancer have been reported a lot, but the data are still conflicting.

Accordingly, the present inventors have conducted studies to develop a material for preventing, alleviating or treating cancer metastasis, thereby completing the present invention.

### DISCLOSURE

### Technical Problem

An object of the present invention is to provide a pharmaceutical composition for preventing or treating cancer containing coffee as an active ingredient.

### Technical Solution

One aspect of the present invention is directed to a pharmaceutical composition for preventing or treating cancer metastasis containing coffee as an active ingredient.

In one embodiment of the present invention, the coffee may be any one or more of coffee beans, a coffee extract, a coffee fraction, and a coffee extraction or fractionation by-product.

In one embodiment of the present invention, the cancer may be any one of liver cancer, colorectal cancer (MC38), lung cancer, pancreatic cancer, breast cancer, rectal cancer, prostate cancer, thyroid cancer, and gastric cancer.

In one embodiment of the present invention, the composition is able to inhibit cancer metastasis, cancer growth, and VCAM1 expression.

In one embodiment of the present invention, the dose of the composition may be 0.2 g to 2 g of the coffee per kg body weight.

### Advantageous Effects

The composition for preventing or treating cancer of the present invention is able to inhibit cancer metastasis, cancer growth, and VCAM1 protein expression, and thus may be used to prevent or treat cancer.

### BRIEF DESCRIPTION OF DRAWINGS

FIG. 1 is a schematic diagram showing an animal experiment process performed in Experimental Example 1.

FIG. 2 shows the results of Experimental Example 2-1. FIG. 2(A) depicts photographs showing cancer tissue metastasized to the liver, after 3 weeks in the seeding phase (administration of coffee for 2 weeks before liver metastasis of colorectal cancer), and FIGS. 2(B) and 2(C) are graphs showing the results of quantifying the cancer tissue.

FIG. 3 shows the results of Experimental Example 2-2. FIG. 3(A) depicts photographs showing cancer tissue metastasized to the liver, after 3 weeks in the soiling phase (administration of coffee for 2 weeks after liver metastasis of colorectal cancer), and FIGS. 3(B) and 3(C) are graphs showing the results of quantifying the cancer tissue.

FIG. 4 shows the results of Experimental Example 2-3. FIG. 4(A) depicts fluorescent photographs showing cancer metastasized to the liver, after 1 day in the seeding phase, and FIGS. 4(B) and 4(C) are graphs showing the results of quantifying the cancer.

FIG. 5 shows the results of Experimental Example 2-4. FIG. 5(A) depicts fluorescent photographs showing the protein expression of VCAM1 as a cancer metastasis promoter, FIG. 5(B) is a graph showing the results of quantifying the protein expression, and FIG. 5(C) is a graph showing the results of quantifying the gene expression.

### BEST MODE

One aspect of the present invention provides a pharmaceutical composition for preventing or treating cancer containing coffee as an active ingredient.

In one embodiment of the present invention, the coffee may be any one or more of coffee beans, a coffee extract, a coffee fraction, and a coffee extraction or fractionation by-product.

In the present invention, the term “extract” is meant to include an extract itself and an extract of any formulation that may be formed using the extract, for example, an extract obtained by extraction of the coffee beans, a dilution or concentrate of the extract, a dried product obtained by drying the extract, a crude product or purified product of the extract, or a mixture thereof.

In the present invention, the term “fraction” refers to a product obtained by a fractionation method of separating a specific component or a specific group from a mixture containing various components. According to one embodiment of the present invention, the coffee extract may be a fractionation product obtained by a solvent fractionation method using a solvent such as n-hexane or ethyl acetate, and examples thereof include both a polar solvent fraction and a non-polar solvent fraction. Specific examples of the fraction include a hexane fraction, an ethyl acetate fraction, and a water fraction.

In the present invention, the term “coffee extraction or fractionation by-product” refers to a substance except for the coffee bean extract or coffee fraction prepared in the process of preparing the coffee bean extract or coffee fraction described above.

In the present invention, the coffee may be specifically a coffee extract, more specifically a water extract of coffee and/or a dried product thereof, most specifically a freeze-dried product of a hot water extract of coffee.

In one embodiment of the present invention, the cancer may be any one of liver cancer, colorectal cancer (MC38), lung cancer, pancreatic cancer, breast cancer, rectal cancer, prostate cancer, thyroid cancer, and gastric cancer, specifically liver cancer, without being limited thereto.

In one embodiment of the present invention, the composition is able to inhibit cancer metastasis, cancer growth, and VCAM1 expression. Specifically, the composition is able to inhibit the development or metastasis of cancer after administration thereof (Experimental Examples 2-1 and 2-3), and the growth of cancer can be inhibited by administration of the composition after the development or metastasis of cancer (Experimental Example 2-2). In addition, the composition is able to inhibit cancer metastasis by inhibiting the expression of cancer metastasis-related VCAM1 protein and/or an mRNA thereof.

In the present invention, the term “prevention” includes any action that inhibits or delays the metastasis and growth of cancer cells by administration of the composition.

In the present invention, the term “treatment” refers to any action that alleviates or beneficially changes symptoms of cancer by administration of the composition.

The pharmaceutical composition for preventing or treating cancer according to the present invention may be composed of only the above active ingredient, but may also further contain various auxiliary additives to improve, specifically, storage stability, processability into formulations, compatibility with other ingredients in the product, and the like. Auxiliary additives that are used include various kinds of additives having different functions, such as a surfactant, an extender, a viscosity modifier, a colorant, and a flavoring agent. Specific examples of the auxiliary additives include glycerin, castor oil, glycine, Tween, dextrin, organic acids (e.g., lactic acid, tartaric acid, adipic acid, succinic acid, citric acid, ascorbic acid, glycolic acid, malic acid, mandelic acid, acetic acid, sorbic acid, benzoic acid, and salicylic acid), antibacterial enzymes, sugars, sugar alcohols, guar gum, cellulose gum, silicone antifoams, and artificial sweeteners.

The pharmaceutical composition for preventing or treating cancer according to the present invention may contain a pharmaceutically effective amount of coffee alone or may further contain one or more pharmaceutically acceptable carriers.

In the present invention, the term “effective amount” means an amount that is very sufficient to deliver desired effects but is small enough to sufficiently prevent serious side effects within the scope of medical judgment. The amount of coffee that is administered in vivo by the composition of the present invention may be appropriately adjusted in consideration of the route of administration and a subject to whom the composition is to be administered.

In addition, the term “pharmaceutically acceptable” as used herein refers to a composition which is physiologically acceptable and, when administered to the human beings, does generally not cause allergic reactions such as gastrointestinal disorders and dizziness, or similar reactions.

The pharmaceutically acceptable carriers are those commonly used for formulation, and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present invention may further contain an excipient, diluent, lubricant, wetting agent, sweetener, flavoring agent, emulsifier, suspending agent, preservative, and the like, in addition to the above components.

The pharmaceutical composition of the present invention may be administered orally or parenterally, and is preferably administered orally. The pharmaceutical composition of the present invention may be formulated in various oral or parenteral dosage forms, without being limited thereto.

Formulations for oral administration include, for example, tablets, pills, hard/soft capsules, solutions, suspensions, emulsifiers, and syrups, granules, elixirs, etc., and these formulations may contain one or more diluents or excipients such as commonly used fillers, extenders, wetting agents, disintegrants, lubricants, binders, and surfactants, in addition to the above-described active 5 ingredient. Examples of disintegrants that may be used include agar, starch, alginic acid or its sodium salt, calcium monohydrogen phosphate anhydrous, and the like, and examples of lubricants that may be used include silica, talc, stearic acid or its magnesium or calcium salt, polyethylene glycol, and the like, and examples of binders that may be used include magnesium aluminum silicate, starch paste, gelatin, tragacanth, methyl cellulose, sodium carboxymethyl cellulose, polyvinylpyrrolidine, low-substituted hydroxypropyl cellulose, and the like. In addition, lactose, dextrose, sucrose, mannitol, sorbitol, cellulose, glycine and the like may be used as diluents, and in some cases, commonly known azeotropic mixtures, absorbents, colorants, flavoring agents, sweeteners, and the like may also be used.

The pharmaceutical composition of the present invention may be administered to a subject one or more times a day. The term “unit dosage form” refers to a physically discrete unit suitable for unitary administration to human subjects and other mammals, wherein each unit contains a suitable pharmaceutical carrier and a predetermined amount of the coffee of the present invention that exhibits a therapeutic effect.

In one embodiment of the present invention, the dose of the composition may be 0.1 g to 2.5 g, specifically 0.2 to 2.3 g, more specifically 2.0 g of the coffee per kg body weight. However, the dose is variable depending on the severity of the patient's intestinal and the microorganism and y active ingredients used. In addition, the total daily dose may be divided into several doses and administered continuously as needed.

### Mode for Invention

Hereinafter, one or more embodiments will be described in more detail with reference to examples. However, these examples are intended to illustrate one or more embodiments, and the scope of the present invention is not limited to these examples.

### Example 1: Preparation of Coffee and Determination of Dosage Concentration

Coffee extract was obtained from Nestle Korea Co., Ltd. The extract is one made by mixing several Arabica species and Robusta species and extracting the mixture with hot water, followed by freeze-drying. The dose of the coffee was divided into a low dose (0.2 g/kg) and a high dose (2 g/kg) based on coffee sticks (1.1 g/stick) sold by Nestle Korea. The dose calculation formula is as follows: 1.1 g/60 kg (1 stick per day for an adult)×12 (mouse body surface area)/60 (adult weight)=about 0.2 g/kg (one cup, low dose), 2 g/kg (ten cups, high dose).

### Experimental Example 1: Animal Experiment Plan and Schedule

C57BL/6 N type mice (8 weeks old, male, 9 to 35 mice) were purchased from Daehan Biolink (ChungBuk, Korea) and allowed access to solid feed (Daehan Biolink) and tap water ad libitum during an adaptation period of 7 days at 22±2° C. and 55±10% relative humidity under a 12-hour light/12-hour dark cycle. After 1 week of adaptation, mice with an average body weight of 20±2 g were randomly divided into groups as follows: a control group; a group to which 0.2 g/kg coffee was administered; and a group to which 2 g/kg coffee was administered. The experimental method is as follows.

### Experimental Example 1-1. Seeding Phase

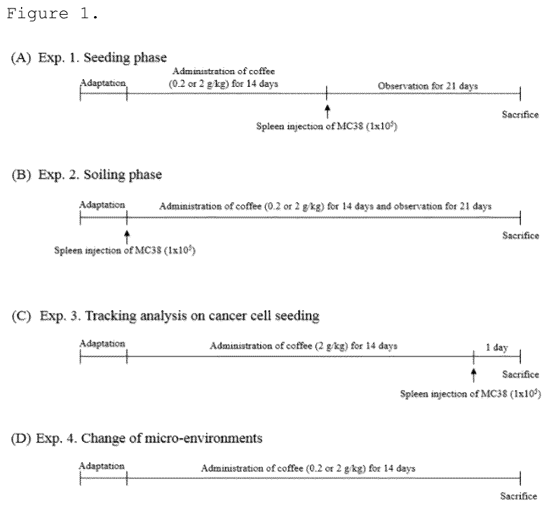

First, 0.2 g/kg coffee, 2 g/kg coffee, or water as a control was orally administered to each mouse group for 14 days. Then, the MC38 cell line (1×10⁵) was injected into the liver t through the spleen of each mouse. 21 days after administration, the mice were sacrificed and the size and number of tumors grown in the liver were measured (FIG. 1A).

### Experimental Example 1-2. Soiling Phase

The MC38 cell line (1×10⁵) was injected into the liver through the spleen of each mouse. After 3 days, 0.2 g/kg coffee, 2 g/kg coffee, or water as a control was orally administered to each mouse group for 14 days. 21 days after administration of the MC38 cell line, the mice were sacrificed and the size and number of tumors grown in the liver were measured (FIG. 1B).

### Experimental Example 1-3. Tracking Analysis on Cancer Cell Seeding

First, 0.2 g/kg coffee, 2 g/kg coffee, or water as a control was orally administered to each mouse group for 14 days. Then, the fluorescently labeled MC38 cell line (1×10⁵) was injected into the liver through the spleen of each mouse. 1 day after administration, the mice were sacrificed and the number of cancer cells in the liver was observed under a fluorescence microscope (FIG. 1C).

### Experimental Example 1-4. Change of Micro-Environments

First, 0.2 g/kg coffee, 2 g/kg coffee, or water as a control was orally administered to each mouse group for 14 days. Then, the mice were sacrificed, and blood and liver tissues were harvested, which were used as evaluation tools for changes of the microenvironments (FIG. 1D).

### Experimental Example 2: Evaluation of Inhibitory Effect of Coffee Against Liver Metastasis of Colorectal Cancer Following Animal Experiments

### Experimental Example 2-1. Evaluation of Effect of Coffee Pre-Administration in Seeding Phase

After the coffee was administered before the cancer cells were administered, whether the microenvironment of the liver tissue was changed by the coffee and how many cancer cells infiltrated the liver tissue were evaluated 3 weeks after administration based on the size and number of tumors grown in the liver.

Specifically, after sacrificing the mice of Experimental Example 1-1, the liver was harvested and weighed, and the number of tumors grown in the liver was measured.

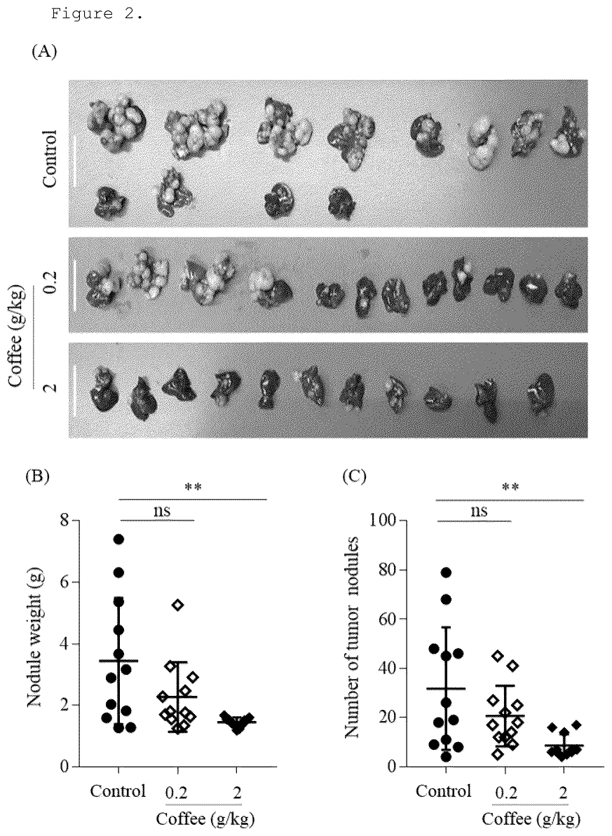

As a result, it was shown that, compared to the control group in terms of the liver weight, the size of tumors grown in the liver did not decrease in the 0.2 g/kg coffee pre-administered group, but significantly decreased in the 2 g/kg coffee pre-administered group (p<0.01, FIG. 2B). The number of tumors also significantly decreased in the 2 g/kg coffee pre-administered group compared to the control group (p<0.01, FIG. 2C). The above results were confirmed again through liver photographs (FIG. 2A).

### Experimental Example 2-2. Evaluation of Effect of Coffee Post-Administration in Soiling Phase

The present inventors examined how much cancer cells grew after adhesion to liver tissue.

Specifically, after sacrificing the mice of Experimental Example 1-2, the liver was harvested and weighed, and the number of tumors grown in the liver was measured.

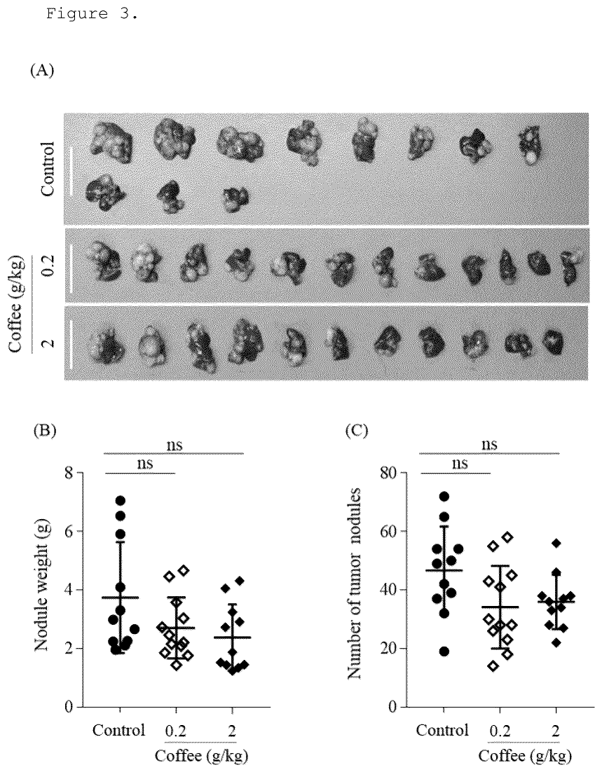

As a result, it was shown that, compared to the control group in terms of the liver weight and the number of tumors grown in the liver, the size and number of tumors in the liver tended to decrease in the 0.2 g/kg and 2 g/kg coffee post-administered groups (p>0.05, FIGS. 3B and 3C). The above results were confirmed through liver photographs (FIG. 3A).

### Experimental Example 2-3. Evaluation of Effect of Coffee Pre-Administration in Initial Seeding Phase

Based on the results of Experimental Example 2-1, whether the microenvironment of the liver tissue was changed by the coffee and how many cancer cells infiltrated the liver tissue were evaluated immediately after seeding based on the number of cancer cells infiltrating the liver.

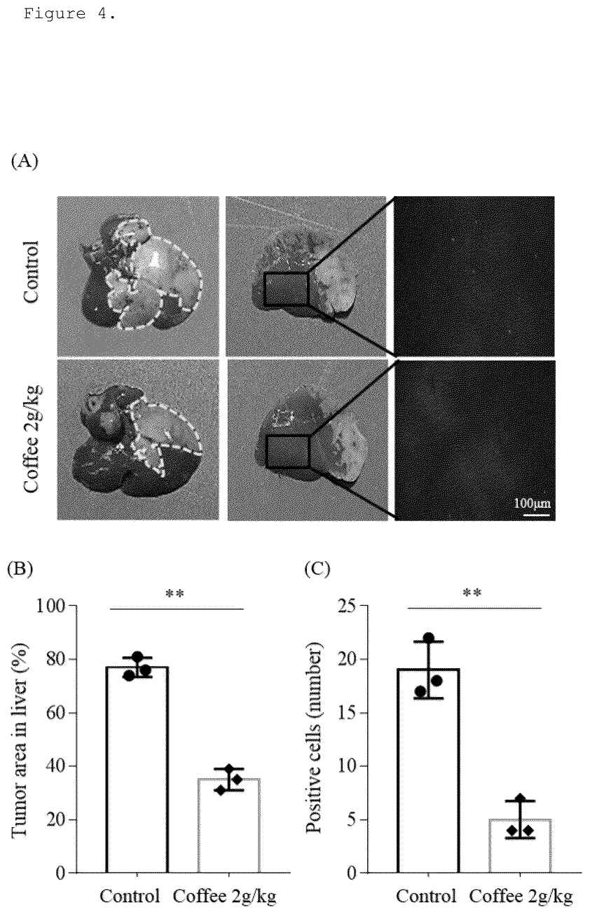

Specifically, after sacrificing the mice of Experimental Examples 1-3, photographs of the liver were taken (FIG. 4A).

As a result, it was shown that, compared to the control group, the area of cancer infiltrating the liver, observed with the naked eye, significantly decreased in the 2 g/kg coffee pre-administered group (p<0.01, FIG. 4B), and the number of fluorescently labeled cells observed under a microscope also significantly decreased in the 2 g/kg coffee pre-administered group (p<0.01, FIG. 4C).

### Experimental Example 2-4. Analysis of Protein and Gene Expression in Liver Following Coffee Pre-Administration in Initial Seeding Phase

In the seeding phase, cancer migrates from blood to tissues. At this time, vascular endothelial cells express adhesion molecules, and cancer cells migrate into tissues through the adhesion molecules.

The present inventors analyzed the protein and gene expression of vascular cell adhesion protein 1 (VCAM1), which is one of the above-described adhesion molecules, following coffee pre-administration in the initial seeding phase.

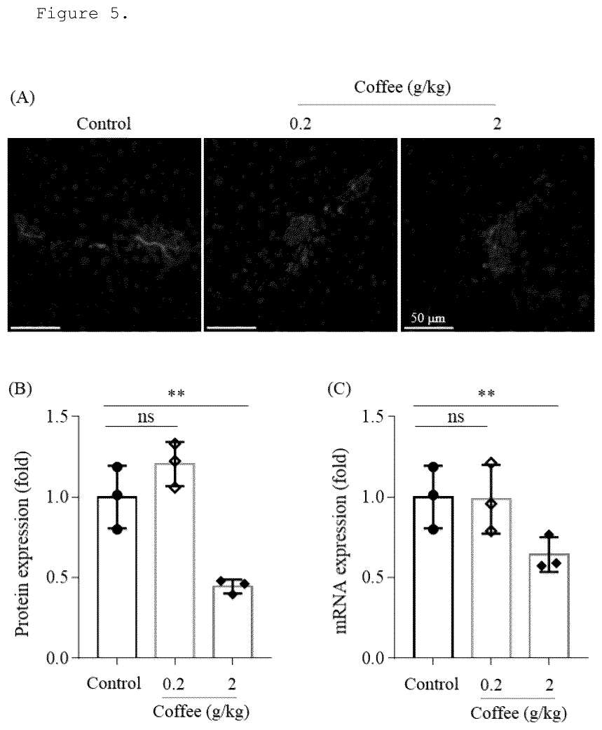

Specifically, protein expression of VCAM1 in the livers of the mice obtained in Experimental Examples 1˜4 was analyzed by immunofluorescence staining (red color, FIG. 5A) and quantified.

As a result, it was shown that, compared to the control group, the protein expression of VCAM1 did not decrease in the 0.2 g/kg coffee pre-administered group, but significantly decreased in the 2 g/kg coffee pre-administered group (p<0.01, [symbol] 5B). In addition, the mRNA expression of VCAM1 also significantly decreased in the 2 g/kg coffee pre-administered group (p<0.01, FIG. 5C).

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. A method for preventing or treating cancer metastasis comprising administering a pharmaceutical composition containing coffee as an active ingredient.

2. The method for preventing or treating cancer metastasis according to claim 1, wherein the coffee is any one or more of coffee beans, a coffee extract, a coffee fraction, and a coffee extraction or fractionation by-product.

3. The method for preventing or treating cancer metastasis according to claim 1, wherein the cancer is any one of liver cancer, colorectal cancer (MC38), lung cancer, pancreatic cancer, breast cancer, rectal cancer, prostate cancer, thyroid cancer, and gastric cancer.

4. The method for preventing or treating cancer metastasis according to claim 1, wherein the composition is able to inhibit cancer metastasis, cancer growth, and VCAM1 expression.

5. The method for preventing or treating cancer metastasis according to claim 1, wherein a dose of the composition is 0.2 g to 2 g of the coffee per kg body weight.
